(19) United States
(12) Patent Application Publication
(10) Pub. No.: US 2004/0176754 A1
(43) Pub. Date: Sep. 9, 2004

(54) Method and device for sensing skin contact

(76) Inventors: Tobin C. Island, Oakland, CA (US); Mark V. Weckwerth, Pleasanton, CA (US); Robert E. Grove, Pleasanton, CA (US)

Correspondence Address: GRAY CARY WARE & FREIDENRICH LLP, 153 TOWNSEND, SUITE 800, SAN FRANCISCO, CA, 94107, US

(21) Appl. No.: 10/794,504

(22) Filed: Mar. 5, 2004

Related U.S. Application Data
(60) Provisional application No. 60/452,591, filed on Mar. 6, 2003.
(60) Provisional application No. 60/456,379, filed on Mar. 20, 2003.
(60) Provisional application No. 60/458,861, filed on Mar. 27, 2003.
(60) Provisional application No. 60/472,056, filed on May 20, 2003.
(60) Provisional application No. 60/456,586, filed on Mar. 21, 2003.

(51) Int. Cl. A61B 18/20

(52) U.S. Cl. 606/9; 607/88; 607/89

(57) ABSTRACT

A skin contact sensor and method are disclosed in a dermatologic treatment device that includes a skin contacting structure, a treatment source capable of being activated to supply a dermatologic treatment through the skin contacting structure. A plurality of sensors are positioned around a periphery of the skin contacting structure, and control circuitry coupled to the plurality of sensors inhibits activation of the dermatologic treatment device unless contact with a compliant surface is sensed. Another embodiment employs a single sensor which is positioned distal to the skin contacting structure so that a non-compliant surface in contact with the skin contacting structure is unable to activate the single sensor.

### PRIORITY

This application claims the benefit of priority under 35 U.S.C. § 19(e) to U.S. provisional patent application Nos. 60/452,591, filed Mar. 6, 2003; 60/456,379, filed Mar. 20, 2003; 60/458,861, filed Mar. 27, 2003; 60/472,056, filed May 20, 2003; and 60/456,586, filed Mar. 21, 2003.

### FIELD OF THE INVENTION

The present invention relates to devices and methods which involve skin contact sensors for dermatologic treatment.

### BACKGROUND OF THE INVENTION

Many skin treatment devices require contact between an active area of the device and the skin for reasons of safety and/or efficacy.

For example, in light-based hair removal systems, the light energy is typically delivered through a cooled transparent surface that makes contact with the skin. In this case, the active area of the device is the cooled, light-emitting surface, and skin contact to this active area is required for at least two reasons: (1) cooling—the cooled surface protects the skin by conducting heat away from the epidermis, and (2) eye safety—contact with the skin eliminates stray light which poses a significant eye hazard. (Some light remits to the environment from outside the active area due to scattering within the skin, but this light poses dramatically less risk than light directly incident upon the eye or directly reflected off the skin surface).

Other examples of treatment devices that require skin contact include (1) devices that require contact only to prevent light leakage, such as a UV illuminator that requires no skin cooling but has a contacting baffle to prevent stray light, or (2) devices that require contact only for their mechanism of action and not to prevent light leakage, such as a thermal heater that delivers a pulse of heat through direct conduction to the skin. Other dermatological devices and methods that involve skin contact include ultrasound and radio frequency applications, such as wrinkle reduction. Some dermatological devices and methods provide skin contact through an interface material, such as ultrasound gel, oil, water, or index matching fluid. It is to be understood that these devices and methods are still considered to be skin contacting for the purposes of this application.

A significant problem for such devices is that the operator may angle or tilt the device's applicator such that it is not perpendicular to the skin. This can create the situation where the entire surface of the active area is not in contact with the skin, and therefore the objective of safety and/or efficacy of the skin contact will not be achieved. This situation is shown graphically in FIG. 1 where an applicator 10 is pressed against a compliant surface 14 that represents skin. The face 11 of the applicator tip 12 represents the active area of the device. As shown in the figure, a non-perpendicular applicator can produce regions where no contact occurs, shown schematically as Region A. Clearly, light leakage could occur from such a region and conductive skin cooling or any other action dependent on contact would not occur or would be less effective.

Another problem for light-based devices is due to eyeglasses. Typical contact sensors would generally sense positive contact if an applicator was applied to a person's eyeglasses, creating a potential for emission directly into the eye that could lead to serious injury or blindness. A similar condition could be created with household window panes or other similar transparent surfaces, whereby a contact sensor could sense contact against the window and light could be dangerously emitted into the ambient environment. It would be desirable, therefore, for a dermatologic contact sensor not to be activated by eyeglasses or similar surfaces.

The mechanical compliance of the surface material (and/or applicator) is an important parameter in these problems. If the material is non-compliant, a non-perpendicular applicator would make contact only upon a line or a point and a large portion of the active area would not be in contact. If the material is very compliant, a non-perpendicular applicator could make contact across the entire active area. Skin has a mechanical compliance that varies due to differences in skin thickness, elasticity, bone backing, and other parameters, but is generally moderately-compliant, such that reasonable levels of applicator angles can indeed produce substantial regions of non-contact for active areas typical of existing devices. This statement is supported by the patient burns that occur occasionally in the light-based hair removal industry; the burns have a shape that indicates a lack of contact cooling across the entire active surface attributed to a non-perpendicular applicator. Furthermore, the fact that skin is moderately-compliant is one parameter that distinguishes skin from eyeglasses, and this parameter could be exploited to make a contact sensor that is immune to eyeglasses or similar hard surfaces.

### CURRENT STATE OF THE ART

Despite the importance of skin contact, existing commercial skin treatment devices do not typically directly sense skin contact. Instead, the systems generally rely on operator training and expertise, which increases the cost of treatments and lowers safety and efficacy (as demonstrated by the burns noted above).

There are, however, various means known in the art to sense skin contact for related devices, including resistive, capacitive, pressure, strain, mechanical, optical, imaging, magnetic, and temperature means.

U.S. Pat. No. 6,508,813 (granted January 2003) to Altshuler describes the use of a temperature sensor near the skin-contacting end of a dermatology device. There may be various controls responsive to the temperature sensor. This patent is presumably the basis of the E-2000 commercial laser system manufactured by Palomar Medical Technologies.

Muller et al. (U.S. Pat. No. 5,360,426, granted November 1994) describe a force-controlled contact applicator for laser radiation, including an element displaceably mounted so as to move in response to contact pressure. A spring may resiliently bias the element in opposition to the contact pressure to define a pre-given force within the displacement range of the element. There may be various controls responsive to the sensor.

U.S. Pat. No. 5,643,252 (granted July 1997) to Waner et al. discloses a laser-based skin perforator that may incorporate a safety interlock. The safety interlock may be a spring-loaded mechanism that is depressed by skin contact to a location where a switch is closed and the laser will initiate a pulse of radiation.

Similarly, Muncheryan (U.S. Pat. No. 3,622,743, granted November 1971) describes a laser-based typography eraser and microwelder that includes a spring-loaded retractable tip that activates the laser through a switch when the tip is depressed onto the working surface.

In U.S. patent application 2003/0032950 (published February 2003) and PCT application WO 02/094116A1 (Published November 2002), Altshuler et al. discuss a variety of skin contact sensors, including optical methods using the treatment beam or a separate light source, electrical contacts to measure resistance or capacitance, and mechanical sensors such as spring-loaded pins or buttons that may be located around the perimeter of an optical element.

In U.S. patent application 2002/0005475 (published January 2002), Zenzie describes a skin contact detecting method and apparatus based upon detecting light at a skin contacting surface. The invention may include a detector for sensing light at the surface and controls responsive to the detector.

A review of the state of the art shows that the existing devices and methods have important deficiencies. In particular, the existing designs do not solve the problem described above where the device applicator is applied at an angle and are not immune to contact by eyeglasses. For example, with the Altshuler temperature sensor, a fraction of the active area may be in contact with the skin and produce a temperature profile indicative of contact, but the signal does not reasonably ensure that the entire active area is in contact. Similarly, spring-loaded mechanical mechanisms, such as described by Waner or Muller, could be activated by contact with eyeglasses and also do not reasonably ensure that the entire active area is in contact. Such designs may allow light leakage, regions of poor contact cooling, and other safety and efficacy concerns associated with lack of skin contact. Furthermore, existing devices and methods are also unnecessarily complex, costly, unreliable, or have other impracticalities. For example, spring-loaded and sliding mechanisms are difficult to clean, are subject to variable friction loads, and add complexity to the assembly.

Thus, there is a clear need for a practical contact sensor for skin treatment devices that would ensure skin contact across the entire active area of the device and would not be activated by eyeglasses and similar hard surfaces. Such an invention would solve a problem of existing methods and devices that occurs when the device applicator is applied at an angle and improve eye safety. Furthermore, such an invention may indeed be a requirement for the expected emerging market of consumer skin treatment devices, as these products cannot rely upon the trained and expert users of physician devices to achieve safety and/or efficacy.

### SUMMARY OF THE INVENTION

The foregoing and other problems and disadvantages of contact sensors in skin treatment devices are overcome by the present invention of a dermatologic treatment device which includes a skin contacting structure, a treatment source capable of being activated to supply a dermatologic treatment through the skin contacting structure, a plurality of sensors around a periphery of the skin contacting structure, and control circuitry coupled to the plurality of sensors and configured to inhibit activation of the dermatologic treatment device unless contact with a compliant surface is sensed.

In one embodiment the treatment source includes a source of electromagnetic radiation, and the skin contacting structure comprises a window through which electromagnetic radiation is emitted. The source of electromagnetic radiation and the dermatologic treatment can be configured to provide hair regrowth inhibition. In such an embodiment, activation of the source of magnetic radiation will be inhibited unless contact with a compliant surface, such as skin, is sensed by way of the sensors.

Other embodiments of the dermatologic treatment device are contemplated in which the treatment source is a source of electromagnetic radiation which is configured for such treatments as acne treatment, photorejuvenation, wrinkle reduction, depigmentation, or repigmentation, and the activation of the source of magnetic radiation is inhibited unless contact with a compliant surface, such as skin, is sensed by way of the sensors.

In further embodiments of the present invention, the ability to sense the presence of a compliant surface is further enhanced by shaping or positioning the skin contacting structure with respect to the sensors so that the sensor activation points are distal from the skin contacting structure by a selected amount. For example, the skin contacting structure can have a surface which is convex in shape so that a non-compliant surface, such as an eyeglass lens, cannot come into contact with the sensors when the skin contacting structure is in contact with the non-compliant surface. An alternative embodiment employs a skin contacting surface which is flat but positions the sensors to be recessed or distal with respect to the skin contacting surface. Another embodiment employs a single sensor which is positioned distal to the skin contacting structure so that a non-compliant surface in contact with the skin contacting structure is unable to activate the single sensor.

In accordance with the present invention, a method for providing a skin contact sensor in a dermatologic treatment device having a skin contacting structure and a treatment source capable of being activated to supply a dermatologic treatment through the skin contacting structure, includes the steps of positioning a plurality of sensors around a periphery of the skin contacting structure, and inhibiting activation of the treatment source unless contact with a compliant surface is indicated by signals from the plurality of sensors. The method can further include the step of configuring the skin contacting structure so that the plurality of sensors is distal from the skin contacting structure by a predetermined amount. The configuring step can include the step of shaping the skin contacting structure to have a convex skin contacting surface.

It is therefore an object of the present invention to provide a skin contact sensor and method suitable for use in dermatologic treatment devices.

It is another object of the present invention to provide a skin contact sensor and method for dermatologic treatment devices in which the skin contact sensor inhibits activation of a treatment source in the device unless contact with a compliant surface is sensed.

It is a further object of the present invention to provide a dermatologic treatment device having a skin contact sensor including a plurality of sensors positioned around a periphery of a skin contacting structure and circuitry coupled to the plurality of sensors and configured to inhibit activation of a treatment source in the device in the presence of a non-compliant surface.

It is still another object of the present invention to provide a skin contact sensor and method for use in dermatologic treatment devices in which a plurality of sensors are positioned around a treatment window and the plurality of sensors are distal to a skin contacting surface of the window by a selected amount.

It is a still further object of the present invention to provide a skin contact sensor configuration and method in a dermatologic treatment device in which a three or more sensors are positioned around a treatment window and a skin-contacting surface of the treatment window is shaped so that the three or more sensors are recessed with respect to the skin-contacting surface by a selected distance.

These and other objectives, advantages and features of the present invention will be more readily understood upon considering the following detailed description of certain preferred embodiments of the present invention, and the accompanying drawings.

### INCORPORATION BY REFERENCE

What follows is a list of citations corresponding to references which are, in addition to those references cited above and below, and including that which is described as background and the invention summary, hereby incorporated by reference into the detailed description of the preferred embodiments below, as disclosing alternative embodiments of elements or features of the preferred embodiments that may not otherwise be set forth in detail below. A single one or a combination of two or more of these references may be consulted to obtain a variation of the elements or features of preferred embodiments described in the detailed description below. Further patent, patent application and non-patent references are cited in the written description and are also incorporated by reference into the preferred embodiment with the same effect as just described with respect to the following references:

U.S. Pat. Nos. 5,360,426; 5,643,252; 3,622,743; 6,508,813;

United States published application nos. 2002/0005475; 2003/0032950;

U.S. provisional patent applications No. 60/451,091, filed Feb. 28, 2003; 60/456,379, filed Mar. 20, 2003; 60/458,861, filed Mar. 27, 2003; 60/472,056, filed May 20, 2003; 60/450,243, filed Feb. 25, 2003; 60/450,598, filed Feb. 26, 2003; 60/452,304, filed Mar. 4, 2003; 60/451,981, filed Mar. 4, 2003; 60/452,591, filed Mar. 6, 2003; and 60/456,586, filed Mar. 21, 2003, all of which are assigned to the assignee of the subject application (collectively, the “Cross-Referenced Provisional Applications”);

United States non-provisional patent application Ser. No. ______, filed Feb. ______, 2004, entitled “Self-Contained Eye-Safe Hair-Regrowth-Inhibition Apparatus And Method,” naming as inventors Tobin C. Island, Robert E. Grove, and Mark V. Weckwerth; Ser. No. ______, filed Feb. ______, 2004, entitled “Eye-Safe Dermatologic Treatment Apparatus And Method,” naming as inventors: Robert E. Grove, Mark V. Weckwerth, Tobin C. Island; and Ser. No. ______, filed Feb. ______, 2004, entitled “Self-Contained, Diode-Laser-Based Dermatologic Treatment Apparatus And Method,” naming as inventors: Mark V. Weckwerth, Tobin C. Island, Robert E. Grove, all of which are assigned to the assignee of the subject application (collectively “the Cross-Referenced Non-Provisional Applications”);

Published PCT application no. WO 02/094116;

Attention is drawn to the aforementioned Cross-Referenced Provisional Applications and Cross-Referenced Non-Provisional Applications by the same inventors of the subject application that disclose various aspects of dermatologic devices. It is clear that one of ordinary skill in the art will recognize that aspects and features disclosed in those applications may be configured so as to be suitable for use with the contact sensor device and method described herein.

### BRIEF DESCRIPTION OF THE DRAWINGS

FIG. 1 is a schematic illustration of an applicator that is angled or tilted with respect to the skin.

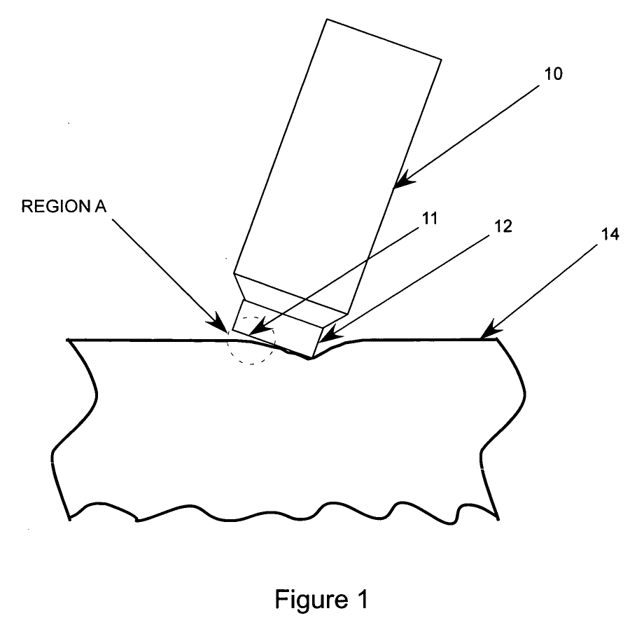

FIGS. 2A and 2B are a schematic illustration of an applicator tip that includes multiple contact sensors arranged around the periphery in accordance with the present invention.

FIG. 3 is a schematic illustration of an applicator tip that includes a convex window and multiple contact sensors in accordance with the present invention.

FIG. 4 is a schematic illustration of an applicator tip that includes a flat window and multiple contact sensors in accordance with the present invention.

FIGS. 5A, 5B and 5C are a schematic illustration of a resilient membrane contact sensor and an assembly in an applicator tip in accordance with the present invention.

### DETAILED DESCRIPTION OF THE PREFERRED EMBODIMENTS

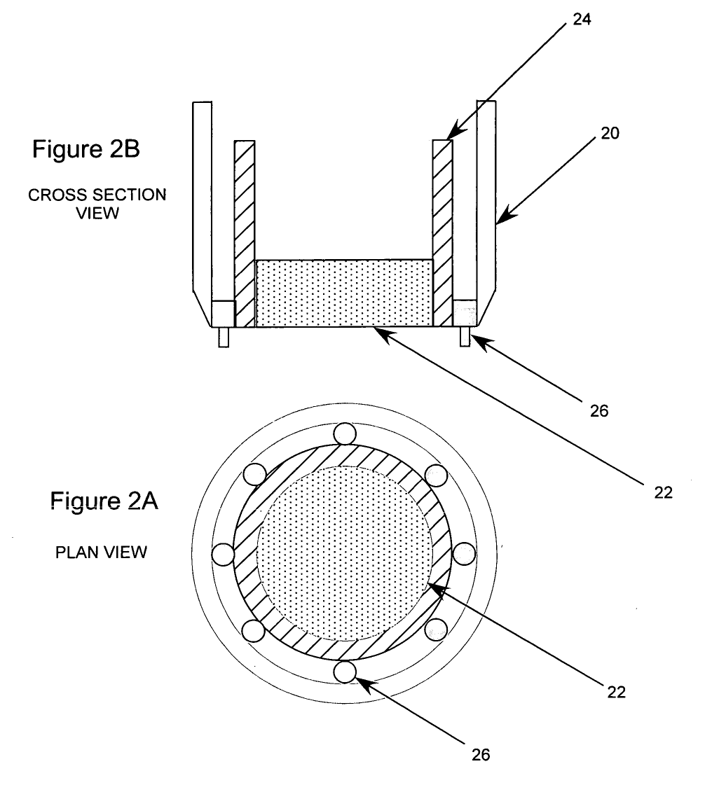

FIGS. 2A and 2B show a first aspect of the invention related to multiple contact sensors arranged around a periphery of a therapeutic surface of a device. In the cross section view, FIG. 2B, housing 20 contains a skin contacting, therapeutic surface 22 attached by a supporting structure 24 (that may serve to cool or heat surface 22) and multiple contact sensors 26. Surface 22 may be a surface emitting light, ultrasound, thermal pulses, radio frequency pulses, or other therapeutic energy. In this example, the contact sensors are shown as mechanical switches with spring-biased actuating pins that depress into the switch body upon contact with skin, but could be any number of sensor types, including electrical contacts to sense resistance or capacitance or temperature sensors. The plan view of FIG. 2A shows eight contact sensors 26 arranged radially around the perimeter of skin-contacting surface 22. The switches can be hard-wire connected in series, such that the device is not considered to be in contact with skin unless all eight switches are “closed”, or could be arranged in series and parallel configurations, or could be sampled by an electronic circuit with a variety of hardware or software algorithms. In practice, the sensor type and properties, the number of sensors, the geometry of the sensor placement, and the electronic circuitry for the sensors would be chosen so as to provide a positive indication of skin contact across the entire surface 22 as required by the use of the device in which the sensor is located.

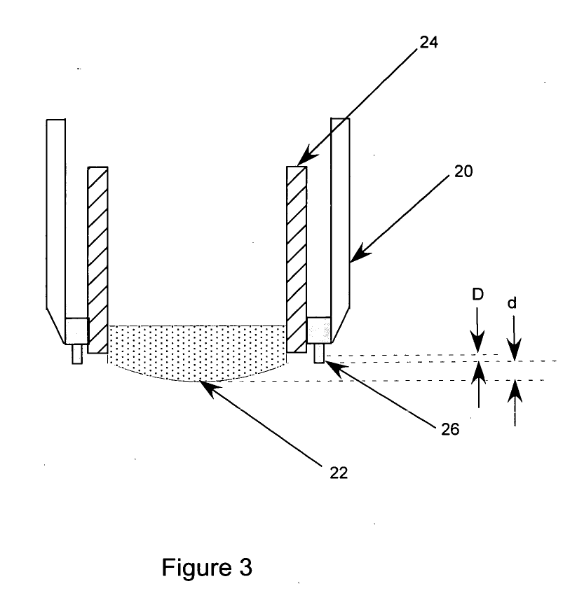

FIG. 3 shows a second aspect of the invention related to contact immunity to eyeglasses and similar non-compliant surfaces. In this figure, housing 20 contains a skin-contacting, therapeutic surface 22 attached by a supporting structure 24 (that may serve to cool or heat surface 22) and multiple contact sensors 26, shown again in this example as mechanical switches with actuation pins. The tips of the actuation pins are recessed a distance “d” from the outermost location of surface 22. Distance “D” represents the distance that the actuation pins travel before the switch changes state. With this geometry, contact with a hard, relatively flat surface such as eyeglasses or plate glass could not activate all of the contact sensors simultaneously. On the other hand, an appropriately compliant material under sufficient pressure could conform to the surface 22 and also depress all of the actuators at least a distance of “D”, thereby indicating positive contact with the compliant material. Such a design provides both a high degree of confidence that the entire active area of the device is in contact with the skin and inhibits undesired activation from contact with eyeglasses or similar surfaces.

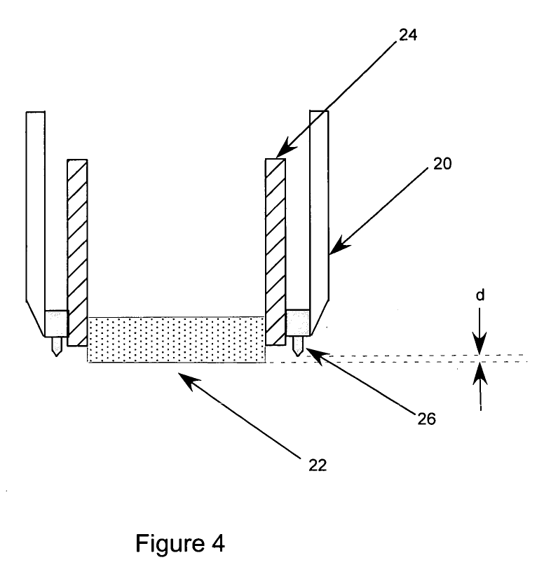

In FIG. 3, a skin-contacting surface 22 is shown as convex but, as shown in FIG. 4, the surface may be flat, or have other geometries. FIG. 4 also shows an example where the sensors are electrical contacts and are located a distance “d” below the skin-contacting surface 22, in order to provide high confidence that the entire surface 22 is in contact with a compliant surface.

Thus, in accordance with the present invention the contact sensors 26 are positioned to have a sensor activation point which can be in the same plane as the skin-contacting surface 22 or, preferably, distal to skin-contacting surface 22, for example from about 0 mm to about 1 mm. More preferably, the sensor activation point is about 0.1 mm to 1 mm distal to the skin-contacting surface. As illustrated in FIGS. 3 and 4, the above can be achieved by selecting the geometries of skin-contacting surface 22 and/or the positioning of the contact sensors 26.

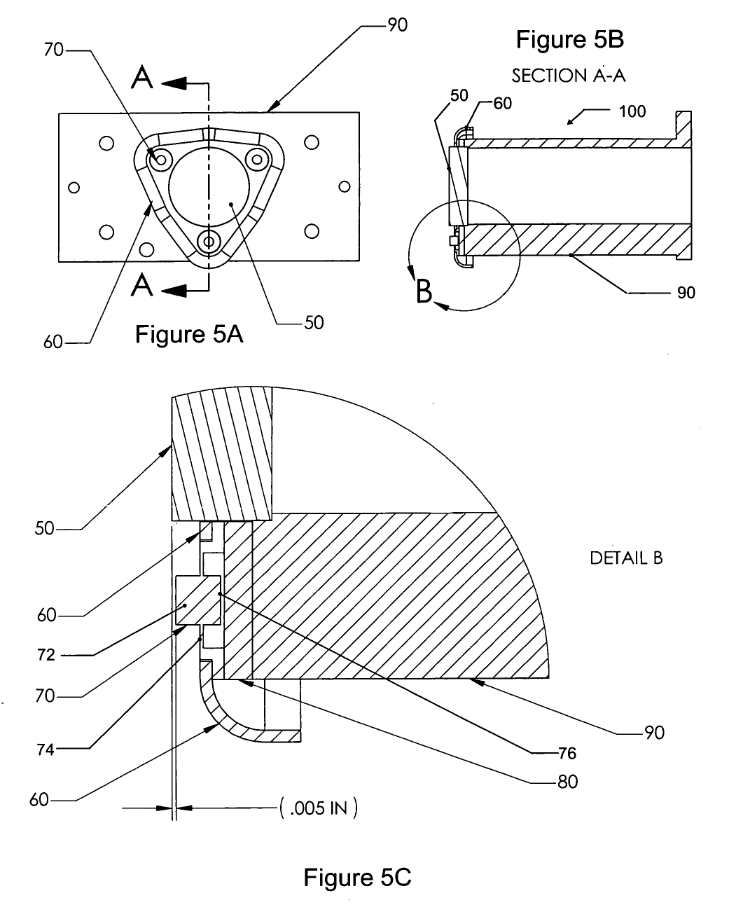

FIGS. 5A, 5B and 5C show a preferred embodiment of the invention. In FIG. 5A, a front view is shown of a dermatologic applicator tip comprising a flat skin-contacting surface 50 surrounded by a bezel 60 and supported by a structure 90. Protruding from the bezel are three mechanical contact sensor “buttons” formed as part of a resilient membrane 70. A cross-section view is shown in FIG. 5B (labeled “SECTION A-A”), and a detailed cross-section view of a portion of the applicator tip is shown in FIG. 5C (labeled “DETAIL B”). Referring to FIG. 5C, resilient membrane 70 is shaped such as to have a protruding button 72 separated from the rest of the membrane by a thin web 74. Upon sufficient force to the top (or outermost surface) of the button 74, the web deforms such that the opposite surface 76 of the button comes into contact with printed circuit board (PCB) 80 which is supported by element 90. The surface of the button that contacts PCB 80 is coated with a conductive ink. PCB 80 has exposed inter-digitated traces located under the button. Normally, the inter-digitated traces are not electrically connected to each other, but when a button is sufficiently depressed, its conductive surface electrically connects the traces, thereby forming a switch.

In a preferred embodiment, the state of each button switch is monitored independently by a microprocessor which has a software algorithm that requires all three switches to be in the “closed” state for the device to be considered in contact. The algorithm preferably also requires that each button switch change state to the “open” state between treatment periods, such as between light-pulses, to assure that the buttons are not permanently in the “closed” state. Contact sensor failure could be detected in this manner. Further details and information about circuitry for interfacing with and processing information from the above sensors, and for implementing control methodologies based on the switch states, suitable for use in the present invention can be found in the above mentioned Cross-Referenced Non-Provisional Applications and the Cross-Referenced Provisional Applications.

Also, in a preferred embodiment, the output for the skin treatment device may be automatically triggered by the contact sensor, improving ease of use and obviating the expense and complication of an additional triggering element, such as a finger trigger. For example, for a hair growth inhibition procedure, a therapeutic light pulse could be automatically initiated upon positive contact. Note that the additional safety provided by ensuring contact across the entire active area of the device and immunity to activation from contact with eyeglasses is an important benefit to automatic firing.

In the preferred embodiment, membrane 70 is made of 40-60 durometer silicone, the button protrudes approximately 0.030 inches above the outermost portion of the bezel 60, the diameter of the button is approximately 0.060 inches, the web thickness is approximately 0.005 inches, the web length is approximately 0.030 inches, and the gap between the traces on PCB 80 and the conductive surface of the button is approximately 0.005 inches. Membrane 70 is bonded to bezel 60 and PCB 80 except in the button regions. Furthermore, in this embodiment the top (or outmost surface) of the button is recessed approximately 0.005 inches from the flat skin-contacting surface 50, which may emit light and may provide heat transfer between the skin and the device. This embodiment results in a very low activation force of less than 0.1 oz per button which can easily be provided by skin, yet has sufficient return force provided by the resilient material to be reliable. The three buttons are sufficiently recessed as to reasonably ensure that the entire skin-contacting surface 50 is in contact while being immune to activation by eyeglasses and other similarly hard, flat surfaces, and yet are reliably triggered by moderately-compliant skin over a wide range of anatomical locations. The button size is large enough to be manufactured with standard techniques and provides sufficient skin contact area, yet is small enough to make for a practical sized applicator tip 100. Furthermore, the embodiment is inexpensive, simple, largely waterproof and immune to dirt and other contaminants, and reliable.

The description above is to be considered one preferred embodiment of the invention. As is clear to one of ordinary skill in the art, numerous other embodiments are possible, and may include at least the following alternative aspects.

Other types of sensors could be used, including sensors that work primarily with electrical means, mechanical means, or optical means, and are fundamentally digital or analog in nature (including strain gages, temperature sensors, capacitive sensors, resistive sensors, or acoustic sensors). Sensor types that provide additional means to discriminate skin from other materials, such as resistive sensors or temperature sensors that could be limited to certain pre-established ranges typical for skin may be even more preferable, but can present other complications such as low signal levels or sensitivity to water films. Another configuration would include using more than one type of contact sensor in a single device, such as combining thermal sensors with mechanical switches.

Various sensor geometries could be used, including varying the number of sensors, the effective size of the sensors, the actuation force or pressure required to produce a state change, the distance the sensor activation point is recessed from the active skin-contacting surface of the device, and other such configurations. In a preferred embodiment of the present invention, the sensor active contact area—the area of the sensor which makes contact with skin or other surface—is less than 5 mm², and more preferably less than 2 mm². Also, preferably, the activation force for each sensor is less one (1) oz, and more preferably between about 0.001 oz to about 0.1 oz.

Likewise, other types of sensor circuitry could be used. The sensor output could be processed purely in hardware, or the device could employ various different software or hardware algorithms to improve safety, reliability, or effectiveness, such as allowing use if three of four buttons indicated contact. Additionally, the circuitry could compare signals from the sensors for various additional purposes, such as to estimate the total heat flux through the contact surface.

While exemplary drawings and specific embodiments of the present invention have been described and illustrated, it is to be understood that that the scope of the present invention is not to be limited to the particular embodiments discussed. Thus, the embodiments shall be regarded as illustrative rather than restrictive, and it should be understood that variations may be made in those embodiments by workers skilled in the arts without departing from the scope of the present invention, as set forth in the appended claims and structural and functional equivalents thereof.

In addition, in methods that may be performed according to preferred embodiments herein and that may have been described above, the operations have been described in selected typographical sequences. However, the sequences have been selected and so ordered for typographical convenience and are not intended to imply any particular order for performing the operations, unless expressly set forth in the claims or as understood by those skilled in the art as being necessary.

We claim:

1. A dermatologic treatment device comprising
a skin contacting structure;
a treatment source capable of being activated to supply a dermatologic treatment through the skin contacting structure;
a plurality of sensors around a periphery of the skin contacting structure; and
control circuitry coupled to the plurality of sensors and configured to inhibit activation of the dermatologic treatment device unless contact with a compliant surface is sensed.

2. The dermatologic treatment device of claim 1, wherein the treatment source includes a source of electromagnetic radiation, and the skin contacting structure comprises a window through which electromagnetic radiation is emitted.

3. The dermatologic treatment device of claim 2, wherein the source of electromagnetic radiation and the dermatologic treatment are configured to provide hair regrowth inhibition.

4. The dermatologic treatment device of claim 2, wherein the source of electromagnetic radiation and the dermatologic treatment are configured to provide acne treatment.

5. The dermatologic treatment device of claim 2, wherein the source of electromagnetic radiation and the dermatologic treatment are configured to provide photorejuvenation.

6. The dermatologic treatment device of claim 2, wherein the source of electromagnetic radiation and the dermatologic treatment are configured to provide wrinkle reduction.

7. The dermatologic treatment device of claim 2, wherein the source of electromagnetic radiation and the dermatologic treatment are configured to provide repigmentation.

8. The dermatologic treatment device of claim 2, wherein the source of electromagnetic radiation and the dermatologic treatment are configured to provide depigmentation.

9. The dermatologic treatment device of claim 1, wherein the treatment source is configured to provide a wrinkle reduction treatment.

10. The dermatologic treatment device of claim 1, wherein the treatment source is configured to provide a depigmentation treatment.

11. The dermatologic treatment device of claim 1, wherein the control circuitry automatically activates the treatment source when contact with a compliant surface is sensed.

12. The dermatologic treatment device of claim 1, wherein the plurality of sensors sense changes in electrical parameters.

13. The dermatologic treatment device of claim 1, wherein the plurality of sensors sense changes in mechanical parameters.

14. The dermatologic treatment device of claim 13, wherein the plurality of sensors include a resilient membrane.

15. The dermatologic treatment device of claim 1, wherein the skin contacting structure has a skin contacting area, and the plurality of sensors are positioned to have a sensor activation point distal to the skin contacting area.

16. A dermatologic treatment device comprising
a window shaped to contact a surface and capable of heat transfer with the surface;
a source of electromagnetic radiation capable of being activated to supply a dermatologic treatment through the window;
one or more heat-transfer elements thermally coupled to the window;
three or more sensors around a periphery of the window; and
control circuitry coupled to the three or more sensors and configured to inhibit activation of the dermatologic treatment device unless contact with a compliant surface is sensed.

17. The dermatologic treatment device of claim 16, wherein the control circuitry automatically activates the source of electromagnetic radiation when contact with a compliant surface is sensed.

18. The dermatologic treatment device of claim 16, wherein the window has a convex outer surface.

19. The dermatologic treatment device of claim 18, wherein the three or more sensors are positioned to have a sensor activation point distal to the window.

20. The dermatologic treatment device of claim 19, wherein the three or more sensors sense changes in electrical parameters.

21. The dermatologic treatment device of claim 19, wherein the three or more sensors include mechanical switches.

22. The dermatologic treatment device of claim 19, wherein the three or more sensors include a resilient membrane.

23. The dermatologic treatment device of claim 19, wherein the control circuitry automatically activates the source of electromagnetic radiation when contact with a compliant surface is sensed.

24. The dermatologic treatment device of claim 16, wherein the window has a flat outer surface.

25. The dermatologic treatment device of claim 24, wherein the three or more sensors are positioned to have a sensor activation point distal to the window.

26. The dermatologic treatment device of claim 25, wherein the three or more sensors sense changes in electrical parameters.

27. The dermatologic treatment device of claim 25, wherein the three or more sensors sense changes in mechanical parameters.

28. The dermatologic treatment device of claim 25, wherein the three or more sensors include a resilient membrane.

29. The dermatologic treatment device of claim 24, wherein the control circuitry automatically activates the source of electromagnetic radiation when contact with a compliant surface is sensed.

30. A dermatologic treatment device comprising
a window shaped to contact a surface;
a source of electromagnetic radiation capable of being activated to supply a dermatologic treatment through the window;
three or more sensors around a periphery of the window and positioned to have a sensor activation point distal to the window; and
control circuitry coupled to the three or more sensors and configured to inhibit activation of the dermatologic treatment device unless contact with a compliant surface is sensed.

31. The dermatologic treatment device of claim 30, wherein the three or more sensors sense changes in electrical parameters.

32. The dermatologic treatment device of claim 30, wherein the three or more sensors sense changes in mechanical parameters.

33. The dermatologic treatment device of claim 32, wherein the three or more sensors include a resilient membrane.

34. The dermatologic treatment device of claim 30, wherein the three or more sensors each have an active contact area less than 5 mm².

35. The dermatologic treatment device of claim 34, wherein the active contact area is less than 2 mm².

36. The dermatologic treatment device of claim 30, wherein the sensor activation point is between zero to 1 mm distal to the window.

37. The dermatologic treatment device of claim 30, wherein the sensor activation point is between 0.1 mm to 1 mm distal to the window.

38. The dermatologic treatment device of claim 30, wherein each of the three or more sensors becomes active at a contact force of between about 0 oz. to about 1 oz.

39. The dermatologic treatment device of claim 30, wherein each of the three or more sensors becomes active at a contact force of between about 0.001 oz to about 0.1 oz.

40. The dermatologic treatment device of claim 30, wherein the window has a convex outer surface.

41. The dermatologic treatment device of claim 30, wherein the window has a flat outer surface.

42. The dermatologic treatment device of claim 30, wherein the control circuitry automatically activates the source of electromagnetic radiation when contact with a compliant surface is sensed.

43. A dermatologic treatment device comprising
a window shaped to contact a surface and capable of heat transfer with the surface;
a source of electromagnetic radiation capable of being activated to supply a dermatologic treatment through the window;
one or more heat-transfer elements thermally coupled to the window;
three or more mechanical sensors around a periphery of the window and positioned to have a sensor activation point distal to the window; and
control circuitry coupled to the three or more sensors and configured to inhibit activation of the dermatologic treatment device unless contact with a compliant surface is sensed.

44. The dermatologic treatment device of claim 43, wherein the three or more sensors include a resilient membrane.

45. The dermatologic treatment device of claim 44, wherein the three or more sensors each have an active contact area less than 5 mm².

46. The dermatologic treatment device of claim 45, wherein the active contact area is less than 2 mm².

47. The dermatologic treatment device of claim 46, wherein the control circuitry automatically activates the source of electromagnetic radiation when contact with a compliant surface is sensed.

48. The dermatologic treatment device of claim 44, wherein the sensor activation point is between zero to 1 mm distal to the window.

49. The dermatologic treatment device of claim 44, wherein the sensor activation point is between 0.1 mm to 1 mm distal to the window.

50. The dermatologic treatment device of claim 49, wherein the control circuitry automatically activates the source of electromagnetic radiation when contact with a compliant surface is sensed.

51. The dermatologic treatment device of claim 44, wherein each of the three or more sensors becomes active at a contact force of between about 0 oz. to about 1 oz.

52. The dermatologic treatment device of claim 44, wherein each of the three or more sensors becomes active at a contact force of between about 0.001 oz to about 0.1 oz.

53. The dermatologic treatment device of claim 52, wherein the control circuitry automatically activates the source of electromagnetic radiation when contact with a compliant surface is sensed.

54. The dermatologic treatment device of claim 44, wherein the window has a convex outer surface.

55. The dermatologic treatment device of claim 44, wherein the window has a flat outer surface.

56. The dermatologic treatment device of claim 43, wherein the control circuitry automatically activates the source of electromagnetic radiation when contact with a compliant surface is sensed.

57. A method for providing a skin contact sensor in a dermatologic treatment device having a skin contacting structure and a treatment source capable of being activated to supply a dermatologic treatment through the skin contacting structure, comprising the steps of
positioning a plurality of sensors around a periphery of the skin contacting structure; and
inhibiting activation of the treatment source unless contact with a compliant surface is indicated by signals from the plurality of sensors.

58. The method of claim 57, further including the step of configuring the skin contacting structure so that the plurality of sensors is distal from the skin contacting structure by a predetermined amount.

59. The method of claim 58, where the configuring step includes the step of shaping the skin contacting structure to have a convex skin contacting surface.

59. The method claim 58, wherein the configuring step includes the step of shaping the skin contacting structure to have a flat skin contacting surface, and further including the step of positioning the active contact areas of the plurality of sensors to be recessed with respect to the flat skin contacting surface.

60. A method for configuring a dermatologic treatment device comprising the steps of
providing a window shaped to contact a surface and capable of heat transfer with the surface;
controllably activating a source of electromagnetic radiation to supply a dermatologic treatment through the window;
thermally coupling one or more heat-transfer elements to the window;
positioning three or more mechanical sensors around a periphery of the window and to have a sensor activation point distal to the window; and
inhibiting activation of the dermatologic treatment device unless contact with a compliant surface is sensed by the three or more sensors.

61. The method of claim 60, further including the step of shaping the window so that a non-complaint surface is blocked from activating the mechanical sensors.

62. The method of claim 61, wherein the shaping step includes the step of forming a convex skin-contacting surface on the window.

63. A dermatologic treatment device comprising
a skin contacting structure;
a treatment source capable of being activated to supply a dermatologic treatment through the skin contacting structure;
a sensor positioned with respect to the skin contacting structure so that a non-compliant surface in contact with the skin contacting structure is unable to activate the sensor; and
control circuitry coupled to the sensor and configured to inhibit activation of the dermatologic treatment device unless contact with a compliant surface is sensed.

64. The dermatologic treatment device of claim 63, wherein the treatment source includes a source of electromagnetic radiation, and the skin contacting structure comprises a window through which electromagnetic radiation is emitted.

65. The dermatologic treatment device of claim 64, wherein the source of electromagnetic radiation and the dermatologic treatment are configured to provide hair regrowth inhibition.

66. The dermatologic treatment device of claim 64, wherein the source of electromagnetic radiation and the dermatologic treatment are configured to provide acne treatment.

67. The dermatologic treatment device of claim 64, wherein the source of electromagnetic radiation and the dermatologic treatment are configured to provide photorejuvenation.

68. The dermatologic treatment device of claim 63, wherein the treatment source is configured to provide a wrinkle reduction treatment.

69. The dermatologic treatment device of claim 63, wherein the control circuitry automatically activates the treatment source when contact with a compliant surface is sensed.

70. The dermatologic treatment device of claim 63, wherein the skin contacting structure has a skin contacting area, and the sensor is positioned to have a sensor activation point distal to the skin contacting area.
